(19) United States
(12) Patent Application Publication
(10) Pub. No.: US 2013/0053850 A1
(43) Pub. Date: Feb. 28, 2013

(54) ORIENTED POLYMER IMPLANTABLE DEVICE AND PROCESS FOR MAKING SAME

(71) Applicants: Joseph DeMeo, Newtown Square, PA (US); Patrick E. Heam, Aston, PA (US); Robert L. McDade, Downingtown, PA (US); Michael J. Popow, Elverson, PA (US)

(72) Inventors: Joseph DeMeo, Newtown Square, PA (US); Patrick E. Heam, Aston, PA (US); Robert L. McDade, Downingtown, PA (US); Michael J. Popow, Elverson, PA (US)

(21) Appl. No.: 13/539,307

(22) Filed: Jun. 30, 2012

Related U.S. Application Data
(63) Continuation of application No. 12/650,053, filed on Dec. 30, 2009.
Application No. 12/650,053 is a continuation-in-part of application No. 12/119,959, filed on May 13, 2008.
Application No. 12/119,959 is a continuation of application No. 10/780,159, filed on Feb. 17, 2004, now Pat. No. 7,378,144.

(51) Int. Cl. A61B 17/68

(52) U.S. Cl. 606/77; 606/60

(57) ABSTRACT

A device is formed by a discontinuous process into a bone screw, plate, or fastener, wherein the device has a degree of polymer alignment and strength, and upon reheating above glass transition temperature of the polymer, the device remains dimensionally stable, as it maintains its dimensions, strength, and degree of polymer orientation. In practice of the present invention, the polymer slug is pressed into the die cavity by the actuation of ram press, causing the slug to conform to the die cavity.

### CROSS-REFERENCE TO RELATED APPLICATIONS

This patent document is a Continuation of copending and commonly owned U.S. patent application Ser. No. 12/650,053, filed Dec. 30, 2009, which is a Continuation-In-Part of copending and commonly owned U.S. patent application Ser. No. 12/119,959, filed on May 13, 2008, which is a Continuation of copending and commonly owned U.S. patent application Ser. No. 10/780,159, filed Feb. 17, 2004, now U.S. Pat. No. 7,378,144, in the names of Joseph DeMeo et al. and entitled “Oriented Polymer Implantable Device and Process For Making Same.” The entire contents of the prior applications are expressly incorporated by reference.

### BACKGROUND OF THE INVENTION

This application relates generally to medical implant devices and their production, specifically relating to the process of manufacturing a polymer tissue and/or bone fixation device, preferably made of a resorbable polymer. The invention more particularly concerns a method of manufacturing a resorbable bone fixation device (e.g., plate, screw, rod, pin, etc.) by forcing a provided polymer slug or billet into a mold while the polymer is in a glass transition state, wherein the manufacturing process creates alignment of the polymeric molecular structure and tailored mechanical properties (e.g., higher strength).

Traditional orthopedic fixation systems typically employ metallic hardware (e.g., plates, screws, rods, and the like) formed of biocompatible, corrosion-resistant metals such as titanium and stainless steel. The metallic implants have often been used because of their high strength; however, because the metallic implants are typically stiffer than bone the metal becomes the primary load-bearing member thereby protecting the bone from stress. This leads to a phenomenon known as “stress shielding”, where bone decreases in density (osteopenia) due to the decrease in load on the bone, as described by Wolff's law. A further disadvantage of metallic hardware is it is often necessary to perform another operation to remove the metal implants after the bone has healed.

The main advantage of metallic plates is that they are strong, tough, and ductile allowing them to be deformed or shaped (e.g., “bent”) at room temperature in the operation room, either by hand or with special instruments, to a form corresponding to the surface topography of bone to be fixed. In this way the plate can be fixed flush on the bone surface to which the plate is applied.

In order to remove the necessity of a second operation and to avoid stress shielding, resorbable implants have been developed to have sufficient strength at the time of implantation and be gradually absorbed by the body as the bone heals over time. These resorbable implants are typically fabricated through standard melt processes. Injection molding, compression molding, and extrusion are melt processes in which a polymer is heated to a highly plastic state and forced to flow under pressure. These processes result in a material having a relaxed orientation or molecular arrangement of the polymer as it cools, and typically does not impart great strength values, such as those required for tissue and/or bone fixation treatments suitable for implantation through surgical techniques (e.g., orthopedic applications). In order to yield the appropriate strength, the size of the implants must be increased which can lead to cosmetic issues (bulges, specifically with maxillofacial plates), anatomical interference issues (such as dysphagia with anterior cervical spine plates or tendon irritation with distal radius plates), and an increase in degradation mass which can cause adverse biological reactions as the polymer is resorbed.

Resorbable implants for orthopedic applications (e.g., maxillofacial and spinal plates) have been manufactured by using the melt processes described above. In order to shape these implants to the desired form corresponding to the surface topography of the bone to be fixed, the plates are often heated, such as by immersion in hot saline, or exposure to heated air, until the polymer achieves a temperature above glass-transition. Once heated, the implant may be shaped by hand and/or with special instruments to the necessary form. Should the polymer implant, without being heated above glass transition, be bent beyond its elastic limit, the polymer will typically fracture upon being bent to the degree often necessary for shaping orthopedic fixation plates for application.

Other common techniques utilized in the past for the production of shaped polymer materials have included, machining (e.g. milling, turning, etc.), and extrusion. Machining a desired shape from a generic slug or billet often results in excessive waste, as the amount of material that is trimmed or cut off in making the final product will be much greater than the amount removed during final machining of a molded or formed polymer material that is shaped nearly to final form. For example, in machining a screw shape, having a head and a threaded body portion, from a slug or billet in the shape of a cylinder, material must be removed to arrive at the diameter of the head. Subsequently, more material must be removed to arrive at the desired diameter for the threaded body portion. This extensive machining creates a great amount of chips or cut dust as waste of the material that is machined off.

Excessive waste of raw material is especially problematic in devices constructed of relatively expensive polymers, such as bioabsorbable polymers and medical grade polymers, as costs are elevated due to the loss of the material, or additional costs are incurred in recapturing and recycling the material. A need exists for a manufacturing technique that results in higher productivity and higher yield than machining

It has long been known that the production of a polymer material having an aligned orientation (i.e., not relaxed) of the polymer molecules or structure typically results in a stronger material. This correlation has been discussed in the prior art, for example, see U.S. Pat. Nos. 3,161,709; 3,422,181; 4,282,277; 4,968, 317; and 5,169,587, where it is described, among other things, that polymer materials may be drawn or extruded to cause the orientation of a semi-crystalline or crystalline polymer structure to become substantially aligned, thereby increasing the mechanical strength of the material.

As discussed in U.S. Pat. No. 4,968,317 issued to Tormala et al., the prior art of using melt molding techniques such as injection molding and extrusion to make resorbable polymer implants results in strength values that are typical of thermoplastic polymers. It is known that the strength and modulus values may be increased by creating a reinforced composite (i.e., incorporating reinforcing fibers), however to achieve satisfactorily large strength values with reinforced composites as implants, the implant must necessarily be large in order to accommodate the stresses placed upon it.

As is known, and is further described by Tormala et al., a technique for the processing of polymer material may utilize mechanical deformation, such as drawing or hydrostatic extrusion, to alter the orientation of the molecular structure of crystalline structure and amorphous structure to a fibrillar state, in order to yield higher strength and elastic modulus values. Tormala et al. describe drawing the material through the extrusion process, resulting in an extruded material that is at least partially fibrillated as the polymer molecules and molecular segments are aligned along the drawing direction. Tormala et al. in U.S. Pat. No. 6,383,187 describe a resorbable screw made of the material described in the U.S. Pat. No. 4,968,317 patent. A need exists for a fibrillar material that may be created in varying cross-sections and diameters, in order to minimize the amount of machining required to finish the product. A further need exists for an implantable device having variable states or degrees of alignment of the polymer molecules. This may be accomplished by manufacturing or processing a material that is formed to final part geometry or near final part geometry of a device or implant, thereby reducing the need for final machining, and also obtaining increased mechanical strengths for implant applications.

In U.S. Patent Application 2003/0146541, Nakamura et al. describe a press molding process for the manufacture of a resorbable polymer bone joining device having molecular orientation. The described process requires imparting the existing molecular orientation, preferably by stretching the primary article along the long axis, then providing the oriented primary article for press molding of the screw head and shank threads. The press molding as applied to the polymer material allows the molecular orientation of the primary article to be substantially maintained. Nakamura et al. do not describe a process for creating a device having variable cross section and variable states of alignment of the polymer molecules, wherein the process of manufacturing the areas with varying cross-sections imparts an increased orientation of the polymer molecules.

In U.S. Patent Application 2003/0006533, Shikinami et al. disclose a twice-forged resorbable polymer material, wherein the polymer molecular orientation is altered by each of the forging processes to create “orientation along a large number of reference axes having different axial directions”. The forging steps applied to the polymer result in the orientation of the polymer molecules to create a room temperature flexible material, capable of withstanding repeated bending without breaking U.S. Patent Application 2003/0006533 does not describe a polymer material that is shaped into varied cross-sections and possessing varied zones of polymer orientation.

In U.S. Pat. No. 6,232,384, Hyon discloses a resorbable bone fixation material comprising a resorbable polymer, hydroxyapatite and an alkaline inorganic compound, wherein the bone fixation material is made by the process of providing a melt with the aforementioned components, molecularly orienting the melt through a molding or extension process and extending and orienting the chain molecules of the polymer. Preferably the molding process is performed through ram or hydrostatic extrusion. Hyon does not describe an implantable material having varied cross-section and varied zones of polymer orientation.

In U.S. Pat. No. 6,503,278, Pohjonen et al. disclose an implantable surgical device made from a resorbable, non-crystalline (i.e., amorphous) polymer. The amorphous material described by Pohjonen et al. is molecularly oriented and reinforced by mechanical deformation. Pohjonen et al. do not describe a polymer implant material having zones of variable states of alignment of the polymer molecules and varying cross section of the material.

In U.S. Pat. No. 5,431,652, Shimamoto et al. disclose a high strength polymer material that is hydrostatically extruded through a die under pressure to reduce voids and to form a resorbable polymer material that retains at least 85% of its strength after 90 days implantation. The material described in the Shimamoto et al. patent does not result in a polymer implant material or implant with complex geometry or variable shape other than the cross section of the die exit, nor does Shimamoto et al. arrive at or describe variable states of alignment of the polymer molecules.

In U.S. Pat. No. 6,511,511, Slivka et al. disclose a polymer implant that is either porous or non-porous, where the material has been reinforced by the addition of oriented fibers. The Slivka devices are made by precipitating the polymer out from a solvent solvating the polymer. The precipitation of the polymer causes a gel formation, which may then be handled and placed in a mold. Slivka et al. do not describe a polymer implant having variable shape and variable states of alignment of the polymer molecules.

The prior art described does not disclose a polymer implantable device having an orientation of the polymer molecules, wherein the shaping process creates zones of varying cross section and orientation.

The prior art describes the manufacture of high-strength, oriented polymer plates. As is typical with high-strength, oriented materials, these plates may be bent to some degree at temperatures below the glass-transition temperature of the polymer and do not exhibit the crazing or fracturing that is seen with typical melt-processed (unoriented) polymer plates. However, it is difficult to bend these oriented materials to precise surface topographies, i.e., to match a bone surface, unless the devices are softened by increasing their temperature above the glass-transition temperature of the polymer. A weakness with prior art oriented polymer materials has been that when exposed to temperatures above the glass-transition temperature, these materials typically transition (relax) to a lower-energy molecular configuration. This relaxation is characterized by a dimensional change in the device and a decrease in the strength of the device. For example, plates fabricated from typical reinforcement methods increase in thickness and decrease in length and/or width upon being heated to temperatures above their glass transition. In addition, the bending strength of the plates decreases due to the loss of molecular orientation caused by the relaxation.

Several resorbable polymers have glass-transition temperatures below body temperature. Due to this property, they are limited in their use as orthopedic implants, such as bone fixation devices. A need exists for a process to increase the strength of the polymers, while at the same time ensuring that they remain dimensionally stable and retain their strength when exposed to temperatures above glass transition, such as in the body.

In U.S. Pat. No. 4,968,317 Tormala et al. describe surgical devices (plates, rods, screws, etc.) composed of resorbable polymers that have been drawn in the solid state. In U.S. Pat. No. 5,227,412 Hyon et al. describe biodegradable and resorbable surgical materials fabricated using solid state drawing, specifically uniaxial stretching. In U.S. Pat. No. 5,431,652 Shimamoto et al. describe bone-treating devices and their manufacturing method, specifically ram extrusion, pull-trusion, and hydrostatic extrusion. In U.S. Pat. No. 6,019,763 Nakamura et al. describe a bone joining device fabricated by drawing a polymer and then pressing it along various axes. In U.S. Pat. No. 6,719,935 Tunc describes a continuous reinforcement process consisting of an extruder, 2 pullers with a heat tunnel in between, and a cutter, where the downstream puller runs at a faster speed than the upstream puller thus drawing the extruded material. The above patents all describe a continuous processing technique, and result in a high-strength, oriented polymeric device. Applicants' experience with oriented polymers produced by continuous processing techniques is that they change dimensionally upon reheating; therefore a need exists for a high-strength, oriented polymeric device that remains stable when heated above its glass-transition temperature.

In U.S. Pat. Nos. 6,221,075 and 6,692,497 Tormala et al. describe a bioabsorbable deformable fixation plate fabricated by methods such as those describe in U.S. Pat. No. 4,968,317. It is claimed that these plates are flexible at one temperature (such as room temperature in an operating room) and maintain the bend at a second temperature (such as body temperature). This is true for most high-strength, oriented materials fabricated from resorbable polymers such as polylactide or polyglycolide, where the glass-transition temperature is greater than body temperature. However, when the temperature is increased past body temperature, typical high-strength, oriented polymeric materials will relax and lose both strength and dimensional stability. Due to the complexity of the bending required for orthopedic plates such as maxillofacial plates and distal radius plates, which have small radii of curvature, the plates must be softened to increase their flexibility by increasing the plate above its glass-transition temperature. A need exists for a high-strength, oriented polymeric device that may be heated beyond its glass-transition temperature yet retains its geometry and strength.

In U.S. Pat Nos. 5,981,619, 6,632,497, and 6,908,582 Shikinami et al. describe a biodegradable and bioabsorbable implant and method for adjusting the shape thereof. These patents describe a multiple “forging” or ram extrusion process, where the material undergoes solid-state deformation along a plurality of axes, thereby creating material with multiple planes of orientation. It is claimed that this material may be deformed within ordinary temperature range and has a shape-keeping ability. However, there is no claim of a shape-keeping ability when heated above the material's glass-transition temperature. In addition, this process requires multiple forging processes in order to achieve the multiple axes of orientation resulting in higher labor costs for producing the device and increased thermal degradation of the polymer.

In U.S. Pat No. 6,755,832 Happonen et al. describe a bone plate with shaping areas to reduce the bend resistance of the plate and allow it to be bent to match the bone geometry more easily. This patent does not claim methods for fabricating high-strength plates or describe high-strength polymeric devices that are stable when heated above their glass-transition temperatures.

In U.S. Pat No. 5,863,297 Walter et al. describe a moldable, hand-shapable biodegradable implant material. However, this patent claims a porous device and does not provide for orienting the polymers to provide the high-strength characteristics of oriented polymer materials.

In U.S. Pat No. 5,204,045 Courval et al. describe a process for extruding polymer shapes with a smooth, unbroken surface. Parts are fabricated using a solid-state extrusion process. However, this patent claims a thin, smooth surface layer created by melting the outer layer of the billet being extruded, whereas the present patent describes material that is heated below its melt temperature and, hence, is not melted. This patent does not describe high-strength polymeric devices that are stable when heated above their glass-transition temperatures.

It is the intent of this invention to overcome these and other shortcomings of the prior art.

### SUMMARY OF THE INVENTION

It is an object of this invention to provide a shaped polymer article having sufficient strength to serve as an implantable tissue or bone fixation device. It is also an object of the invention to provide a polymer medical device with increased mechanical properties, resulting from an oriented polymer structure. Furthermore, it is another object of the invention to provide an oriented polymer device that is dimensionally stable when heated above the glass transition, as it is able to be heated above a glass transition temperature of the polymer and maintain the geometry and strength, without relaxation of the oriented polymer structure.

It is another object of the invention to provide a method of manufacturing the implantable device by a process that results in polymer orientation. The degree of polymer orientation has a correlation with the physical properties (e.g., strength, elasticity, etc.) of the material. Higher strength may be achieved by providing higher degree of polymer orientation. In an embodiment, the implantable device features varying zones of polymer orientation, induced by the manufacturing process.

The non-continuous or discontinuous processing of the polymer leads to a reduction in cross-section as it is processed, thereby creating orientation of the polymer, and the resulting product may be heated above the glass transition temperature of the polymer and remains dimensionally stable, without losing dimensional integrity or strength upon being reheated.

In one embodiment, a polymer slug is driven into a die cavity tooling to form an implantable device, having varied cross section and varied degree of polymer orientation.

In an embodiment of the process, the device is formed into a bone screw or fastener, wherein the head has a degree of polymer alignment and strength, and wherein the shank has a higher degree of polymer alignment and strength.

In another embodiment, the device is formed as a rod, pin, or plate, and may be of various cross-sectional profiles, including round, oval, rectangular or irregular in cross-section. The polymer material is typically oriented uniaxially, or where the device has a bend, (e.g., an L-bend bone plate), the orientation of the polymer molecules would follow the contours of the device, and are oriented along a bent axis formed as a consequence of the bend in the device (e.g., L-bend plate, or bent rod).

The process of practicing the one embodiment of the invention (as will be further explained), in its basic form, involves the steps of:
a) providing a polymer slug, die cavity tooling, and ram press, wherein said die cavity tooling defines a die shape;
b) placing said polymer slug between said ram press and die cavity tooling;
c) actuating said ram press in order to apply pressure upon said slug, wherein the polymer slug, while being pressed, is preferably at a temperature above the glass transition temperature of the polymer and below the melting temperature of the polymer, such that the pressing forces said slug to conform to said die shape, wherein said slug is formed into a device comprising zones of variable alignment of the polymer structure, and zones of varying cross-section;
d) removing said device from said die cavity tooling; and optionally,
e) shaping the device to the finished product, the shaping may be performed by a machining procedure, a compression molding procedure or other techniques known in the art.

The process of practicing another embodiment of the invention (as will be further explained), in its basic form involves the steps of:
a) providing a polymer slug, die cavity tooling, and ram press, wherein said die cavity tooling defines a die shape;
b) placing said polymer slug between said ram press and die cavity tooling;
c) actuating said ram press in order to apply pressure upon said slug, wherein the polymer slug, while being pressed, is preferably at a temperature above the glass transition temperature of the polymer and below the melting temperature of the polymer, such that the pressing forces said slug to conform to said die shape;
d) removing said device from said die cavity tooling;
e) heating said device above the glass transition temperature of the polymer, such that the device may be shaped by hand, and further wherein the device is dimensionally stable upon being reheated, and in the heated state maintaining the geometry, strength and orientation of the polymer; and optionally between steps d and e;
f) machining the device to a shape, through a machining procedure, a compression molding procedure or other techniques known in the art.

### DESCRIPTION OF THE DRAWINGS

FIG. 1: Depiction of cylindrical polymer slug, billet, or blank suitable for use in the various embodiments of the invention.

FIG. 2A and 2B: Depictions of alternate shapes of polymer slugs, billets, or blanks

FIG. 3A and 3B: Depictions of polymer slugs, billets, or blanks having complex internal (3A) or external (3B) geometry.

FIG. 4A and 4B: Cross sectional depictions of die tooling arrangements suitable for use in various embodiments of the invention.

FIG. 5A and 5B: Depictions of press ram component having complex external (5A) or internal (5B) geometry.

FIG. 6: Cross sectional depiction of die tooling arrangement having a hollow core forming ejector pin.

FIG. 7: Cross sectional depiction of die tooling arrangement having a solid tip forming ejector pin

FIG. 8: Cross sectional depiction of a multi component die cavity tooling.

FIG. 9: Cross sectional depiction of die tooling arrangement having multiple reductions in cross section—one in the barrel component and one in the die cavity component.

FIG. 10: Cross sectional depiction of die tooling arrangement for creating a bent axis bone fixation device.

FIG. 11A-F: Depictions of typical bone plate geometries.

FIG. 12A and 12B: Depictions of cranio-maxillofacial plates (12A) and cervical spine plates (12B).

FIG. 13A and 13B: Depiction of the effect of temperatures greater than the material glass transition temperature on high-strength, polymeric materials, contrasting the behavior of an embodiment of the present invention with that of the prior art oriented polymer materials.

### DETAILED DESCRIPTION OF PREFERRED EMBODIMENTS

One embodiment of the invention consists of a method for producing a surgical polymer implant, such as a tissue fixation device, or a bone fixation or treating device. The implant may be formed in any shape suitable for implantation into the living being and may be fastened into or onto tissue or bone (e.g. a screw, pin, rod, nail, plate, staple, suture anchor or in the form of a similar type fastener or related component.)

In practice of various embodiments, the polymer is formed as a polymer slug that has been extruded, injection molded, self-oriented or otherwise formed into a solid or near solid mass, preferably cylindrical or rectangular in geometry. However, the slug itself can consist of final part geometry prior to forming. Die tooling is heated until a desired temperature is reached, preferably above the glass-transition temperature (Tg) of the material, but below the melting temperature (Tm), and/or for a desired duration. Typically the slug will be within the barrel, and dry, rather than constantly surrounded by fluid or pressure medium as with hydrostatic extrusion. The slug is then pressed by a ram into the cavity portion of the die tooling and the slug takes the shape of the cavity or preform. The geometry of the die cavity may be round, oval, rectangular, or irregular in cross-section to produce pins, plates, bar stock, or other complex devices. The geometry of the die cavity can promote reduction in one leg dimension (of a rectangular slug for example) while stabalizing the other dimension resulting in uniaxial (along force) orientation. The geometry of the die cavity can also promote reduction in both dimensions of the slug resulting in biaxial (along force axis and transverse to force) orientation through one press operation. Multiple press operations can be performed to further orient uniaxially or achieve biaxial orientation of previously uniaxially (and/or biaxial) oriented part by now applying force transverse to original force direction. The die cavity can be designed to provide a portion or all of the final part geometry and to require minimum material removal to complete the fabrication of the final bone treating device. The die cavity may provide for defining a split in the oriented polymer, subsequently a later bending operation may be employed to form an X- or Y-plate. The die cavity may bend in relationship to the pressing axis to allow the forming of an L-plate. The pressed material does not require a pull-off force and does not exit the cavity portion of the die tooling, but is ejected or removed after proper forming and cooling.

Alternatively, the melt-processing step used to fabricate the slug may be incorporated into the pressing/strengthening operation. For example, rather than allowing an injection molded slug to cool to ambient conditions and then reheating it during the pressing operation, the slug could be allowed to cool to a temperature between Tg and Tm in the mold and then be pressed into the plate geometry. This would significantly decrease the cycle time required to make the devices and remove the need for using two separate pieces of equipment. In addition, this would allow for the pressing of more complex geometries (such as threaded parts, L-plates, and X-plates) and allow the final part geometry to be formed, thus removing secondary operations such as machining or contouring.

The high-strength, oriented material from the pressing process may be in the final device geometry, or may undergo secondary operation(s) such as turning, milling, compression molding, and other processes familiar to those skilled in the art. The secondary operation may also consist of bending the polymer device into a geometry matching the anatomical location where it would be implanted. Due to the unique properties of the material this bending may be performed at temperatures higher than the glass-transition temperature of the polymer in order to achieve more complex curvatures, such as those required for distal radius and maxillofacial plates.

In an embodiment, the bone treating device or implant processed through the methods described herein consists of a bioabsorbable polymeric material or matrix. In an alternative embodiment, the polymeric material of the implant may be non-resorbable. The polymer may feature a semi-crystalline, crystalline or amorphous structure. A semi-crystalline or crystalline structure polymer material features an arrangement of the polymer molecules in three-dimensional spherulitic structures and may further feature lamellae, a folded crystalline structure. The amorphous polymer structure generally lacks the lamellae found in the crystalline and semi-crystalline polymer structures. The polymer matrix material may be composed of a polymer; alternatively the material may comprise a copolymer or a mixture thereof.

The preferred, and most widely used bioabsorbable polymers to be processed through the application of this invention consist of poly(lactic acid) or PLA, poly(glycolic acid) or PGA, their copolymers and stereocopolymers such as poly(glycolide-co-L-lactide) or PGA/PLLA, or Poly-DL-lactide (DLPLA), but are not limited to these preferred or widely used materials. Other resorbable and non-resorbable polymer materials may be suitable for practicing this invention. Examples of resorbable polymers that can be used to form the device are shown in following Table 1. These materials are only representative of the materials and combinations of materials, which can be used in the practice of the current invention.

TABLE 1
Examples of Bioresorbable Polymers for Construction
of the Device of the Current Invention

Aliphatic polyesters
Bioglass
Cellulose
Chitin
Collagen
Copolymers of glycolide
Copolymers of lactide
Elastin
Fibrin
Glycolide/l-lactide copolymers (PGA/PLLA)
Glycolide/trimethylene carbonate copolymers (PGA/TMC)
Hydrogel
Lactide/tetramethylglycolide copolymers
Lactide/trimethylene carbonate copolymers
Lactide/ε-caprolactone copolymers
Lactide/σ-valerolactone copolymers
L-lactide/dl-lactide copolymers
Methyl methacrylate-N-vinyl pyrrolidone copolymers
Modified proteins
Nylon-2
PHBA/γ-hydroxyvalerate copolymers (PHBA/HVA)
PLA/polyethylene oxide copolymers
PLA-polyethylene oxide (PELA)
Poly (amino acids)
Poly (trimethylene carbonates)
Poly hydroxyalkanoate polymers (PHA)
Poly(alklyene oxalates)
Poly(butylene diglycolate)
Poly(hydroxy butyrate) (PHB)
Poly(n-vinyl pyrrolidone)
Poly(ortho esters)
Polyalkyl-2-cyanoacrylates
Polyanhydrides
Polycyanoacrylates
Polydepsipeptides
Polydihydropyrans
Poly-dl-lactide (PDLLA)
Polyesteramides
Polyesters of oxalic acid
Polyglycolide (PGA)
Polyiminocarbonates
Polylactides (PLA)
Poly-l-lactide (PLLA)
Polyorthoesters
Poly-p-dioxanone (PDO)
Polypeptides
Polyphosphazenes
Polysaccharides
Polyurethanes (PU)
Polyvinyl alcohol (PVA)
Poly-β- hydroxypropionate (PHPA)
Poly-β-hydroxybutyrate (PBA)
Poly-σ-valerolactone
Poly-β-alkanoic acids
Poly-β-malic acid (PMLA)
Poly-ε-caprolactone (PCL)
Pseudo-Poly(Amino Acids)
Starch
Trimethylene carbonate (TMC)
Tyrosine based polymers

The appropriate polymer matrix or material to be processed in practicing the various embodiments herein may be determined by several factors, including, but not limited to, the desired mechanical and material properties, the surgical application for which the implant device is being produced, and the desired degradation rate of the device in its final application.

The previously mentioned polymeric materials may also be compounded with one or more additive materials. The additive materials may serve various functions, including, but not limited to, serving to reinforce the polymer matrix material, and serving to deliver therapy or beneficial agents to the body. Examples of reinforcing additive materials include ceramics (e.g., hydroxyapatite, tricalcium phosphate (TCP), etc.), fibrous materials (e.g., fibers, whiskers, threads, yarns, meshes, nets, weaves, etc.), or particulates (e.g., microspheres, microparticles, beads, etc.) In those embodiments where at least one fibrous reinforcement is incorporated, the reinforcing fiber may be in any suitable form (e.g., chopped, short, long, continuous, individual, bundled, weaved, etc.) The reinforcing additive material may be comprised of similar or different material than the polymer matrix material. Suitable reinforcement material may include the previously mentioned and most widely used bioabsorbable polymers, the resorbable polymers of Table 1 above, their copolymers and their stereocopolymers, as well as reinforcement materials such as ceramics, metals and bioactive glasses and their compounds. Reinforcement material may be non-bioabsorbable material, and may also be used in conjunction with a bioabsorbable polymer matrix material and be processed through the method of the present invention to form a bone-treating device. Other non-limiting examples of suitable materials that may be added to the polymer material are listed in Table 2.

TABLE 2
Reinforcing Materials suitable for use in the Present Invention

Alginate
Calcium
Calcium Phosphates
Ceramics
Chitosan
Cyanoacrylate
Collagen
Dacron
Demineralized bone
Elastin
Fibrin
Gelatin
Glass
Gold
Hyaluronic acid
Hydrogels
Hydroxy apatite
Hydroxyethyl methacrylate
Hyaluronic Acid
Nitinol
Oxidized regenerated cellulose
Phosphate glasses
Polyethylene glycol
Polyester
Polysaccharides
Polyvinyl alcohol
Radiopacifiers
Salts
Silicone
Silk
Steel (e.g. Stainless Steel)
Synthetic polymers
Titanium

The additive materials may also comprise biologically active agents (e.g., therapeutics, beneficial agents, drugs, etc.) that are delivered to the living being upon implantation of the device. The additive material may comprise a substance that serves to encourage tissue ingrowth into the device (e.g., TCP, hydroxyapatite, etc.) The additive materials may also serve as a drug delivery mechanism, wherein a biologically active agent is coated onto or mixed with the polymeric material. Alternatively, the biologically active agent may be coated onto or contained within other additive material that is then added to the polymer. The therapy delivery may occur rapidly once implanted (as in the case of a surface coating), or alternatively, longer-term drug delivery is contemplated and may be achieved, where the drug delivery occurs for all or a portion of the duration of the implant's degradation. Examples of biologically active agents that may be delivered in the device are shown in following Table 3. These materials are only representative of the classes or groups of materials and combinations of materials, which can be used in the practice of the current invention, although some specific examples are given.

TABLE 3
Examples of Biological Active Ingredients

Adenovirus with or without genetic material
Alcohol
Amino Acids

L-Arginine

Angiogenic agents
Angiotensin Converting Enzyme Inhibitors (ACE inhibitors)
Angiotensin II antagonists
Anti-angiogenic agents
Antiarrhythmics
Anti-bacterial agents
Antibiotics

Erythromycin
Penicillin

Anti-coagulants

Heparin

Anti-growth factors
Anti-inflammatory agents

Dexamethasone
Aspirin
Hydrocortisone

Antioxidants
Anti-platelet agents

Forskolin
GP IIb-IIIa inhibitors

eptifibatide

Anti-proliferation agents

Rho Kinase Inhibitors
(+)-trans-4-(1-aminoethyl)-1-(4-pyridylcarbamoyl)
cyclohexane

Anti-rejection agents

Rapamycin

Anti-restenosis agents

Adenosine A_{2A} receptor agonists

Antisense
Antispasm agents

Lidocaine
Nitroglycerin
Nicarpidine

Anti-thrombogenic agents

Argatroban
Fondaparinux
Hirudin
GP IIb/IIIa inhibitors

Anti-viral drugs
Arteriogenesis agents

acidic fibroblast growth factor (aFGF)
angiogenin
angiotropin
basic fibroblast growth factor (bFGF)
Bone morphogenic proteins (BMP)
epidermal growth factor (EGF)
fibrin
granulocyte-macrophage colony stimulating factor (GM-CSF)
hepatocyte growth factor (HGF)
HIF-1
insulin growth factor-1 (IGF-1)
interleukin-8 (IL-8)
MAC-1
nicotinamide
platelet-derived endothelial cell growth factor (PD-ECGF)
platelet-derived growth factor (PDGF)
transforming growth factors alpha & beta (TGF-.alpha., TGF-beta.)
tumor necrosis factor alpha (TNF-.alpha.)
vascular endothelial growth factor (VEGF)
vascular permeability factor (VPF)

Bacteria
Beta blocker
Blood clotting factor
Bone morphogenic proteins (BMP)
Calcium channel blockers
Carcinogens
Cells
Chemotherapeutic agents

Ceramide
Taxol
Cisplatin

Cholesterol reducers
Chondroitin
Collagen Inhibitors
Colony stimulating factors
Coumadin
Cytokines prostaglandins
Dentin
Etretinate
Genetic material
Glucosamine
Glycosaminoglycans
GP IIb/IIIa inhibitors

L-703,081

Granulocyte-macrophage colony stimulating factor (GM-CSF)
Growth factor antagonists or inhibitors
Growth factors

Bone morphogenic proteins (BMPs)
Core binding factor A
Endothelial Cell Growth Factor (ECGF)
Epidermal growth factor (EGF)
Fibroblast Growth Factors (FGF)
Hepatocyte growth factor (HGF)
Insulin-like Growth Factors (e.g. IGF-I)
Nerve growth factor (NGF)
Platelet Derived Growth Factor (PDGF)
Recombinant NGF (rhNGF)
Tissue necrosis factor (TNF)
Transforming growth factors alpha (TGF-alpha)
Transforming growth factors beta (TGF-beta)
Vascular Endothelial Growth Factor (VEGF)
Vascular permeability factor (UPF)
Acidic fibroblast growth factor (aFGF)
Basic fibroblast growth factor (bFGF)
Epidermal growth factor (EGF)
Hepatocyte growth factor (HGF)
Insulin growth factor-1 (IGF-1)
Platelet-derived endothelial cell growth factor (PD-ECGF)
Tumor necrosis factor alpha (TNF-.alpha.)

Growth hormones
Heparin sulfate proteoglycan
HMC-CoA reductase inhibitors (statins)
Hormones

Erythropoietin

Immoxidal
Immunosuppressant agents
inflammatory mediator
Insulin
Interleukins
Interlukin-8 (IL-8)
Interlukins
Lipid lowering agents
Lipo-proteins
Low-molecular weight heparin
Lymphocites
Lysine
MAC-1
Methylation inhibitors
Morphogens
Nitric oxide (NO)
Nucleotides
Peptides
Polyphenol
PR39
Proteins
Prostaglandins
Proteoglycans

Perlecan

Radioactive materials

Iodine - 125
Iodine - 131
Iridium - 192
Palladium 103

Radio-pharmaceuticals
Secondary Messengers

Ceramide

Somatomedins
Statins
Stem Cells
Steroids
Thrombin
Thrombin inhibitor
Thrombolytics
Ticlid
Tyrosine kinase Inhibitors

ST638
AG-17

Vasodilators

Histamine
Forskolin
Nitroglycerin

Vitamins

E
C

Yeast
Ziyphi fructus

The inclusion of groups and subgroups in Table 3 is exemplary and for convenience only. The grouping does not indicate a preferred use or limitation on use of any drug therein. That is, the groupings are for reference only and not meant to be limiting in any way (e.g., it is recognized that the Taxol formulations are used for chemotherapeutic applications as well as for anti-restenotic coatings). Additionally, the table is not exhaustive, as many other drugs and drug groups are contemplated for use in the current embodiments. There are naturally occurring and synthesized forms of many therapies, both existing and under development, and the table is meant to include both forms.

The additive materials may also comprise plasticizers or other materials to provide desirable application properties to the final implant device. Plasticizers or materials that enhance the malleability of the material may allow the processing of the material of the present invention to occur at lower temperatures, providing various benefits (e.g., reduced polymer and additive material breakdown, reduced cooling times, reduced costs, increased productivity, increased polymer chain alignment, etc.).

The following description with reference to the associated figures describes the features of the present invention, wherein like numbers refer to like components.

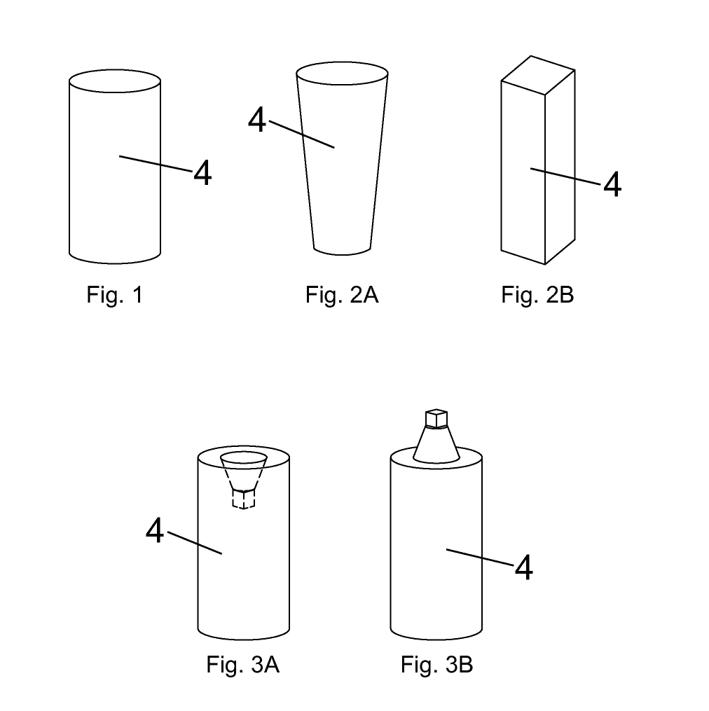

In one embodiment, the invention consists of a method for producing a surgical implant, such as a tissue fixation device, or a bone-treating device, which begins with a provided mass of polymer material called a slug or billet of determinate length. With reference to FIGS. 1, 2A and 2B, the slug of material 4 may be provided having an initial shape or geometry. Preferably, the slug 4 is provided in a simple cylindrical form as shown in FIG. 1, although the slug may be provided in other general shapes, for example, as shown by the alternative slug configurations depicted in FIGS. 2A and 2B.

As can be seen in FIGS. 3A and 3B, the slug 4 may also be provided having a section of more complex geometry, internally and/or externally of the predominate general slug shape. This complex geometry included in the slug may take on the form of geometry that is indicative of the final bone treating device or implant, as can be seen in FIGS. 3A and B. FIG. 3B depicts an example of complex external geometry on a predominately simple cylindrical slug, while FIG. 3A depicts an example of complex internal geometry on a similar cylindrical slug. The complex geometry may be any additional formation than would occur with a general shaped slug in a simple shape (e.g., cylinder, box, conical, etc.)

The complex geometry may be incorporated into the slug through typical melt processing techniques such as injection molding or through traditional machining techniques or alternatively through the method of this present invention. The complex geometries shown in FIGS. 3A and 3B may be final device geometry that is maintained throughout the processing method of the device and such complex geometry in this example could be used as the interface between the final device and the surgical instrument, a driver of a fastener for example. Complex geometry is not limited to the designs shown in FIGS. 3A and 3B, but particular to the geometry of the final implant or device and the extent of feasibility with the processing method described in the present invention.

The slug or billet 4 is described as having a determinate length in that the length and subsequent mass of the slug has been determined and based on the final implant, tissue fixation device or bone treating device to result from the method and tooling utilized and described in the present invention.

The raw material for the provided slug material can be processed and formed through standard manufacturing techniques known in the art, including, but not limited to, traditional melt processes for thermoplastics (e.g., injection molding, single screw extrusion, twin screw extrusion, compression molding, etc., and combinations thereof), as well as through the method of this present invention. Techniques utilized for manufacturing a slug may impart orientation to the polymer structure, as has been discussed earlier, with reference to U.S. Pat. No. 4,968,317. The creation or increase of orientation in the polymer structure results in a stronger material, relative to a similar polymer material lacking equivalent orientation. The preferred material for the provided slug will have at least some orientation, such as a polymer slug material that has been processed through an extrusion process, which inherently creates a degree of molecular orientation. An alternate embodiment may provide a semi or randomly oriented polymer slug material, such as that resulting from injection molding, which offers limited preferred orientation and is heavily dependant upon tooling design and process conditions. However, melt processes not resulting in highly oriented material, such as injection molding, offer advantages that may be necessary in terms of incorporating complex geometry in the slug as shown in FIGS. 3A and 3B. The provided slug material may also be machined to desired geometry and/or tolerances through typical machining techniques following initial typical melt processing. Independent of the degree of molecular orientation of the beginning slug or the method used for fabricating the beginning slug, the final material or device formed by the method of the present invention will result in improved orientation in comparison to the originally provided slug or billet.

The material of the provided slug is processed through the practice of the various embodiments of the present invention to arrive at the final desired implant, tissue fixation device or bone treating device, therefore, any additive materials added to the provided polymer slug are incorporated into the final product of the invention. For example, a fiber reinforced slug results in a fiber reinforced implantable device, similarly, a slug incorporating drug therapy measures will result in an implant incorporating drug therapy measures.

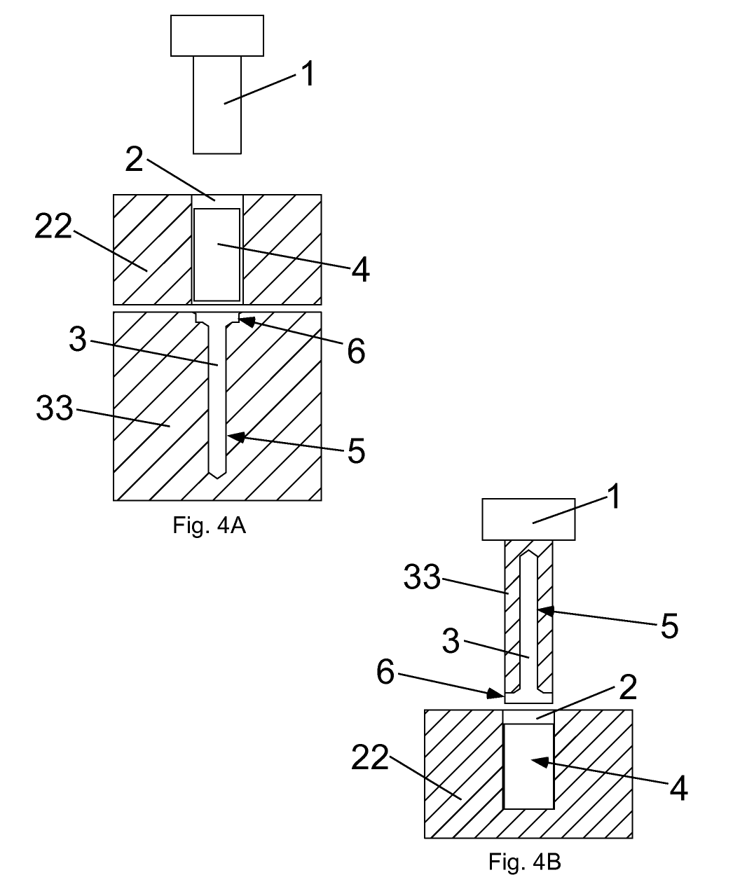

With reference to FIG. 4A, one arrangement of the tooling used for the method of the present invention includes a press ram 1, a barrel 2 or similar holding and/or heating chamber as defined by barrel tooling 22, and a die cavity 3 defined by die cavity tooling 33. The slug 4 is placed in the barrel portion 2 of the barrel tooling 22. The barrel tooling 22 may be a separate component that has been affixed to the die cavity tooling 33 or alternatively may be an integral one-piece design comprising both the barrel tooling 22 and the die cavity tooling 33.

In an alternate, and fundamentally reversed arrangement depicted by FIG. 4B, the die cavity tooling 33, defining the die cavity 3 is operationally attached to the press ram 1. The actuation of the press ram 1 drives the die cavity tooling against the polymer slug 4, contained within the barrel 2, as defined by the barrel tooling 22.

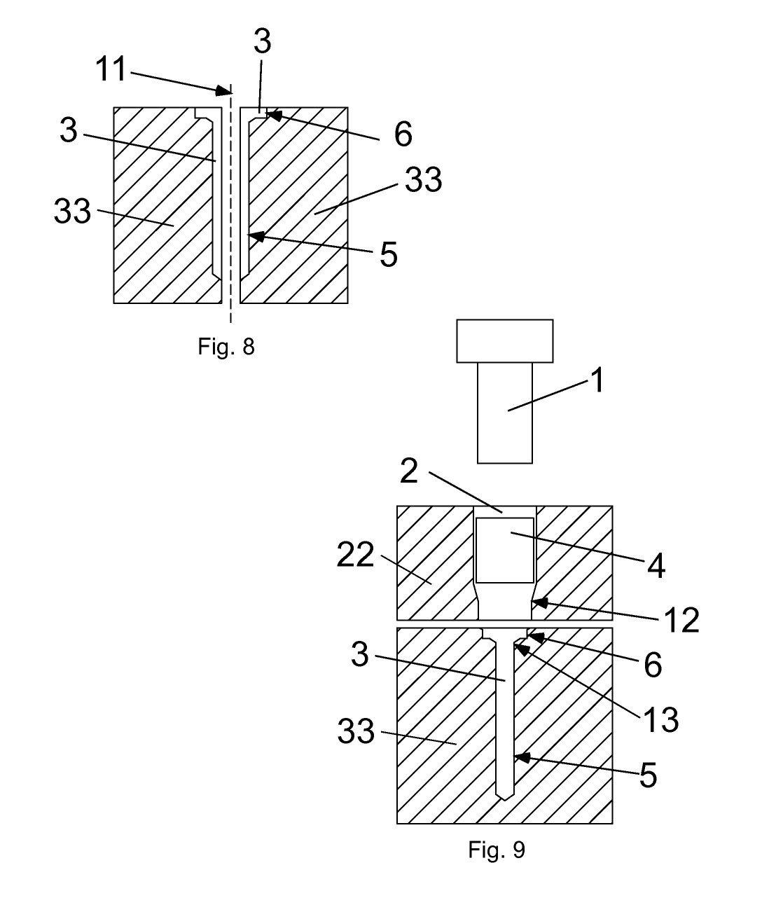

The barrel tooling 22 and die cavity tooling 33 shown in FIGS. 4A and 4B are individually depicted as single piece tooling, respectively forming the barrel geometry 2 and the die cavity geometry 3. It is recognized the particular construction of the barrel tooling 22 and die cavity tooling 33 may beneficially comprise multiple and separable components, particularly a two piece or multiple piece design in which it is preferable, but not necessary, for any parting line of tooling to run parallel with the longitudinal axis of the formed bone treating device or implant. This is particularly true from threaded devices and complex plates that cannot be ejected by typical linear methods. FIG. 8 depicts a cross-sectional view of an exemplary separable, two-piece die cavity tooling 33 consisting of separable die cavity 3 with the parting line 11 of the tooling running parallel with the longitudinal axis of the formed device with device shank 5 and device head 6.

Referring again to the tooling arrangement depicted by FIG. 4A, but applicable to other described embodiments as well, the barrel 2 formed by the barrel tooling 22 should preferably mimic the outside geometry of the slug 4 to be placed within the barrel, though not necessarily. Furthermore, the barrel tooling 22 and die cavity tooling 33 are preferably temperature controlled, incorporating a mechanism to provide heating and/or cooling (not shown). This is to allow proper heat transfer from the barrel tooling 22 to the slug 4. In operation, the slug 4 within the barrel 3 may be heated to a temperature between the glass transition temperature and melting temperature (as in a semi-crystalline polymer) of the material comprising the slug 4 or as applicable based on the material of the slug. The barrel 2 and barrel tooling 22 are heated to this desired temperature either prior to the slug 4 being placed in the barrel 2 or after the slug is placed in the barrel. Alternatively, the processing method for producing the final device also allows for the slug 4 to be heated to a temperature, again between the glass transition temperature and the melting point temperature of the slug material, prior to being placed in the barrel 2. In this case, the barrel may also be pre-heated.

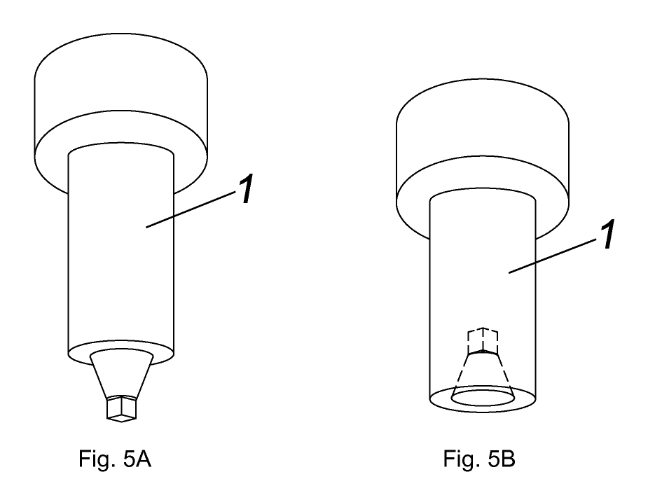

In an embodiment, a temperature gradient extending from the barrel 2 and the slug 4 to the die cavity 3 may be induced. The maximum and minimum temperature within this temperature gradient is preferably maintained between the glass transition temperature of the slug material and the melting temperature of the slug material. It is recognized there may be benefit in temperature set points that are (at least temporarily) somewhat higher or lower than the recorded glass transition and melting temperatures of the polymer, in order to account for heat transfer properties, or to intentionally derive a localized temperature variation. This temperature gradient may consist of a higher temperature at the barrel 2 and slug 4 location than at the die cavity 3 or with the gradient reversed, in which the highest temperature of the temperature gradient exists at the die cavity 3. In this embodiment, the surgical device or implant may have been processed by the method of the present invention at different temperatures along the length of the device. The temperature gradient when processing the material may influence the degree of orientation in the polymer, thereby increasing the mechanical properties along the longitudinal direction of the final surgical implant, tissue fixation device or bone treating device. Following heating of the slug 4 to the desired temperature and/or for the desired duration, the slug is driven by the actuation of press ram 1 into the die cavity 3 portion of the die cavity tooling 33. In a preferred embodiment, the geometry of the end of the press ram 1 in contact with the slug 4 is formed as a flat surface; however, the end may alternatively possess internal and external complex geometry. Complex geometry for the press ram 1 may include external complex geometry as shown in FIG. 5A, or internal complex geometry as shown in FIG. 5B. Either external or internal complex geometry may mimic geometry of the final device and cause the final device to be formed into the slug 4 during pressing. For example, the geometry shown in FIGS. 5A and 5B may form final bone treating device geometry that is used at the interface of the device and a surgical instrument (e.g. a driver of a fastener). Alternatively, the complex geometry shown in FIGS. 5A and 5B may inversely correspond to the complex geometry that is already present in the provided slug as previously discussed with reference to FIGS. 3A and 3B.

The ram 1 may or may not be pre-heated prior to pressing the slug 4. The ram may be driven by typical mechanical means known in the art (e.g., hydraulic, electric, rack & pinion etc.) However, the control and/or variability of speed, positioning, force and dwell may be varied to determine the mechanical and polymer alignment properties of the final part (i.e., the implantable device), and are essential in forming a final implant, tissue fixation device or bone treating device per the method of the present invention. In one embodiment, the actuation of the ram 1 forces the slug 4 into a dry cavity 3, or alternatively, the pressing of the slug may employ lubrication in order to facilitate the flow of the polymer slug 4 into the cavity 3.

In an alternate embodiment, the implant device may be formed by a similar discontinuous process as described above, however relying on hydrostatic pressure (not shown), wherein the actuation of the ram exerts pressure upon a fluid surrounding the slug in the barrel, forcing the slug into the die cavity. As is known in the art, one of the benefits of hydrostatic extrusion is the lubrication afforded by the non-compressible medium surrounding the slug. The device manufactured in the practice of the present invention features varied zones of polymer alignment. This zone variation occurs due to differences in how some areas of the slug 4 undergo deformation in conforming to the die cavity 3 as the ram exerts pressure, resulting in greater elongation and accordingly greater alignment in some areas, while other regions of the slug experience less deformation and therefore feature less alignment.

With reference again to FIG. 4A, the die cavity portion 3 of the die cavity tooling 33 consists of geometry in part or in full of the final bone treating device or implant to be formed. For example, where the implant to be manufactured is a tissue or bone fastener, the die cavity tooling 33 may consist of the shank diameter 5 of the bone fastener and also the head geometry 6 of the bone fastener device. The die cavity 3 consists of reduction in cross sections from one final part geometry to the next. For example, the die cavity depicted in FIG. 4A varies in cross section from the bone fastener head diameter 6 to the shank diameter 5 of the bone fastener form. The reduction in cross section affects the mechanical deformation and further orients the polymer molecules and molecular segments, thereby resulting in increased mechanical properties such as shear and bend resistance in the desired location. This occurs as the polymer slug material 4 that is driven into the shank diameter portion 5 of the die cavity 3 undergoes significantly more deformation and elongation in extending into the shank area, thereby creating significant alignment of the polymer molecules, when compared to the slug 4 material that is formed into the head portion 6 of the die cavity 3, where less deformation and elongation is required, resulting in significantly less reorientation of the polymer molecules. The desired location for increased mechanical properties such as shear and bend resistance, in this example, is the shank diameter 5 of a bone fastener.

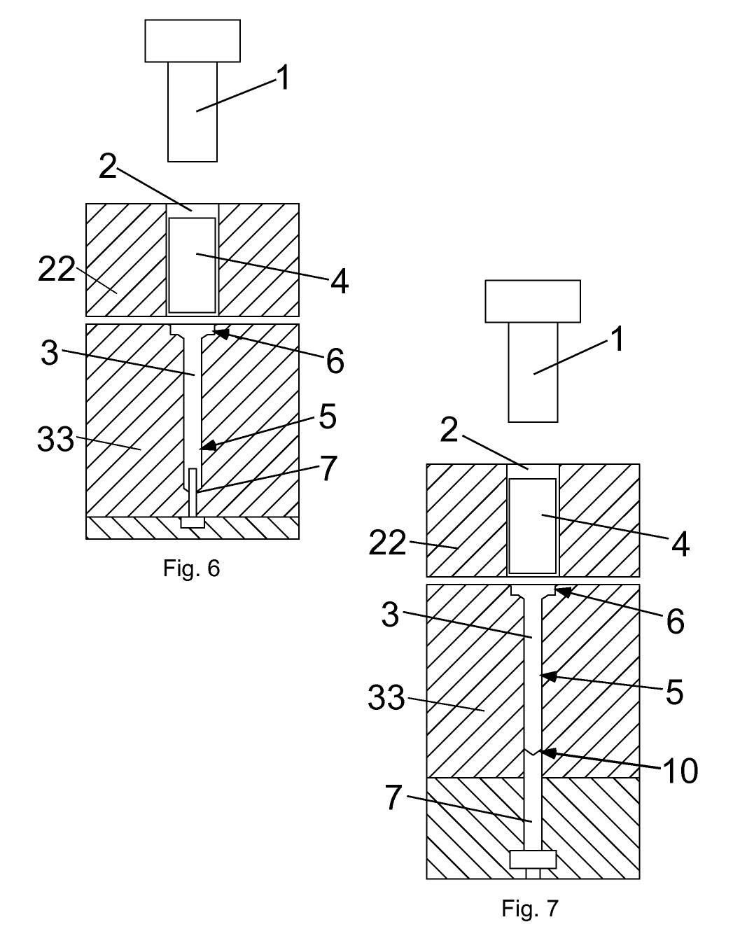

In practice of the present invention, the polymer slug 4 is pressed into the die cavity 3 by the actuation of ram press 1, causing the slug to conform to, and completely fill, the die cavity, or alternatively to at least partially fill the die cavity. The cavity may be a substantially enclosed area defined by the die cavity tooling 33 having only one opening for the introduction and removal of the polymer material (as depicted by the die cavity 3 of FIG. 4A). In another embodiment, the die tooling may feature a second opening away from the ram press 1 to allow for the introduction of an ejection device or pin penetrating through the die cavity tooling, as can be seen in FIGS. 6 and 7.

The ejection device of FIG. 6 features a pin 7 that extends through the die cavity tooling, and extends into the die cavity 3. In this embodiment, the ejection pin further serves to add to the geometry of the final device (e.g., by adding complex geometry as described above). In the example depicted in FIG. 6, the pin 7 may serve to create a slot or a hollow core in the device, created as the pressed polymer slug material surrounds the protruding pin or coring.

In another embodiment, the ejection device depicted in FIG. 7 may serve as a temporarily present die cavity closure, until ejection of the bone treating device is required. Ejection or removal of the bone treating device is preferably performed following proper cooling in the die cavity. The ejection device 7 may optionally consist of geometry 10 particular to the final bone treating device or implant. In the embodiment depicted by FIG. 7, the ejection pin 7 consists of geometry specific to the tip of a bone treating fastener. The ejection pin 7 may be mechanically actuated such that it may reciprocate in order to effect the ejection of the polymer component from the tooling. In this embodiment, the die tooling may provide for a slot to allow the ejection pin to reciprocate.

The reduction or variation in cross-section and the inducing of zones of variable alignment through the pressing method described in the present invention does not need to only take place in the die cavity tooling 33 and die cavity 3, as has been previously described. In an alternative embodiment depicted by FIG. 9, the mechanical strengths of the shaped polymer material may further be increased by continuing to add step-downs in cross-section or increasing the number of variations in cross-section that further align the polymer molecular structure. This may be defined or described as double or multiple-pressing and may take place within either the barrel 2 of the barrel tooling 22, the cavity 3 of the cavity tooling 33, or both. FIG. 9 depicts an example of multiple reductions in cross section further aligning the polymeric molecular structure and obtaining a near net or final shape bone treating device or implant with varying zones or degrees of alignment. In FIG. 9, for example, but not limited to this location, the multiple reductions in cross section take place in both the barrel 2 of the barrel tooling 22 and also the die cavity 3 of the die cavity tooling 33. The locations of the reductions in cross-section and subsequent varying zones of alignment are shown by 12 and 13.

Alternatively, a way to increase the number of reductions in cross section and continue to increase the subsequent mechanical properties is to obtain an implant device through the method of the present invention and to repeat the method of the present invention one or more additional times. This is also an opportunity to not only continue to reduce the cross-section through the pressing operation and increase mechanical properties, but also to continue to add different geometry through the use of different tooling components (e.g., press ram 1, die cavity 3, etc.), the application of which may continue to accomplish a near net shape of the final bone treating device or implant and further reduce and/or eliminate subsequent machining or related processes.

After pressing per the method of the present invention, the device or implant may be cooled in the die components, either under pressure from the ram or another source, or alternatively the implant may be cooled after release of the pressure. Cooling may be controlled by providing for at least one cooling rate, and may vary locally within the die components, and/or temporally. The various cooling rates may be employed as required with respect to the material and design to be cooled.

The implant material, while still in the die cavity 3, may further be re-heated between the glass transition temperature (or thereabouts), and the melt temperature (or thereabouts), of the material and then the cooling process, either with or without pressure, and at one or multiple cooling rates, may be employed, as described above. This heating and/or cooling cycling may be employed as required with respect to the material, the design, and the advantages and/or disadvantages that such heating and/or cooling cycling may have on the final desired properties. For example, an amorphous material may require a different cooling rate(s) and/or a different temperature set point during a re-heating cycle than might a partially crystalline material to gain desired strength increases due to molecular aligning of the respective polymer structure.

In an embodiment, all stages in the manufacturing of the polymer implant device, from slug placement, ram pressing, slug forming within the die cavity, and ejection, are along a common longitudinal axis, which in the case of the simple cylindrical geometry shown in FIG. 1 is the axis of molecular orientation. Similarly, where the processing is to result in a plate, when viewed in cross-section similar to the dimension of FIG. 4, but with the added dimension of depth, where the plate would extend in an axis perpendicular to the cross-sectional plane, the axis of molecular orientation would be along a plane defined by the longitudinal axis, and aligned with the flow of polymer within the die cavity. In the case of a device that has a straight axis, this axis of molecular orientation typically will correspond with the direction of the pressing.

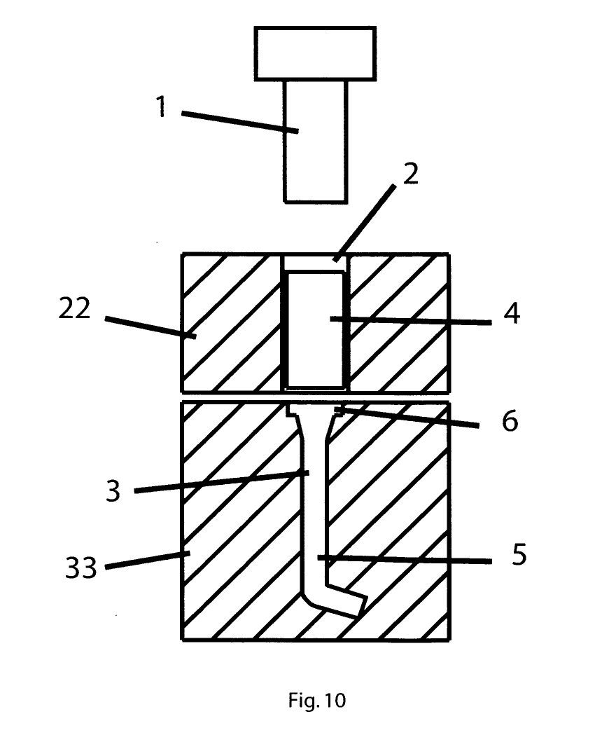

In a similar embodiment to that immediately above, the manufacturing may largely be along a common longitudinal axis, however, within the die cavity, the tooling may provide a complex geometry so as to bend the flow of ram pressed polymer, thus creating a bend in the axis of molecular orientation of the polymer, which has been teamed a bent axis, indicating that the axis has one or more bends to the axis along a dimension of the device. Furthermore, there may be multiple bends forming a variety of shapes and curves in the device. An example of a bent axis die tooling is depicted in tooling arrangement of FIG. 10. This depiction is of a cross-section of a tooling arrangement, and the part produced by such tooling may be in the form of a bent rod or pin, or alternatively a bent plate, such as an L-bend plate. This is particularly useful for complex geometry plates, where such a bent axis would be useful in implantation, for example mandible plates and clavicle plates.

In an alternate embodiment, a technique avoids the need to cool and reheat the slug, as the ram pressing may take place while the slug is still at an elevated temperature (above ambient) due to the manufacturing of the slug or billet. In this embodiment, the slug or billet 4 is formed in the barrel section 2 through standard manufacturing techniques known in the art, including, but not limited to, traditional melt processes for thermoplastics (e.g., injection molding, single screw extrusion, twin screw extrusion, compression molding, etc., and combinations thereof), as well as through the method of this present invention. The slug 4 within the barrel 2 is allowed to cool to the appropriate temperature, preferably between the glass transition temperature and melting temperature (as in a semi-crystalline polymer) of the material comprising the slug 4 or as applicable based on the material of the slug. Following cooling of the slug 4 to the desired temperature and/or for the desired duration, the slug is driven by the actuation of press ram 1 into the die cavity 3 portion of the die cavity tooling 33. In a preferred embodiment, the geometry of the end of the press ram 1 in contact with the slug 4 is formed as a flat surface; however, the end may alternatively possess internal and external complex geometry. After pressing per the methods of the present invention, the device or implant may be cooled in the die components, either under pressure from the ram or another source, or alternatively the implant may be cooled after release of the pressure. Cooling may be controlled by providing for at least one cooling rate, and may vary locally within the die components, and/or temporally. The various cooling rates may be employed as required with respect to the material and design to be cooled. The high-strength, oriented device may then be ejected or removed from the die tooling. This process may be employed in the manufacture of any of the shapes contemplated herein, including rods, and plates.

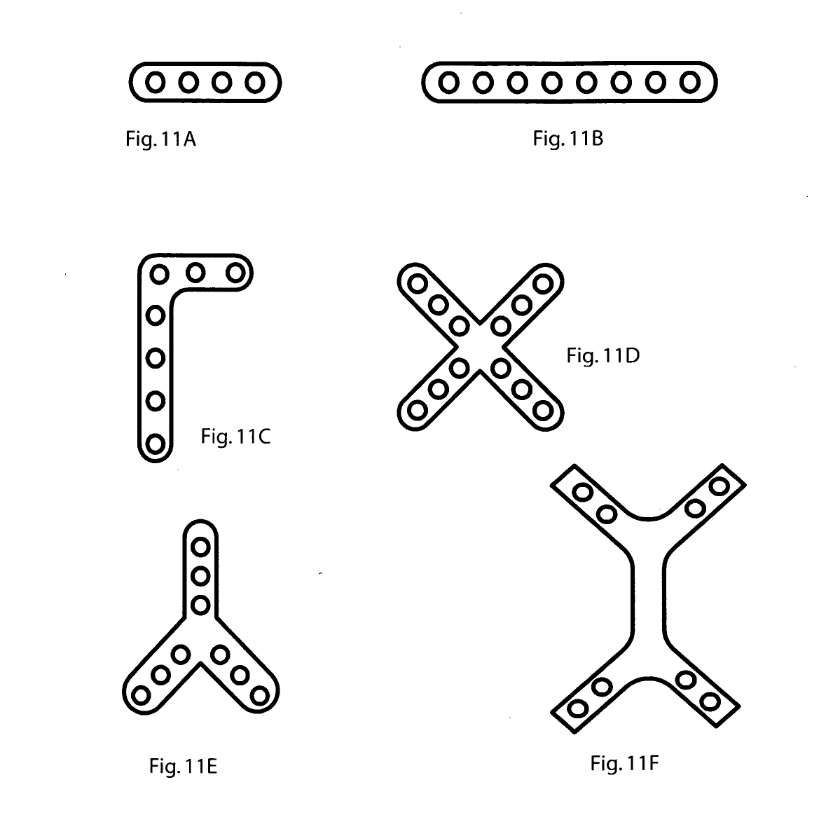

As depicted in FIGS. 11A-F, the finished device may be a plate of various geometries. FIGS. 11A and 11B depict straight plates of various lengths. FIG. 11C shows an L-plate, such as that which may be formed using the tooling depicted in FIG. 10. FIGS. 11D, 11E, and 11F depict complex X- and Y-plates that may be formed using the processes described in the present invention.

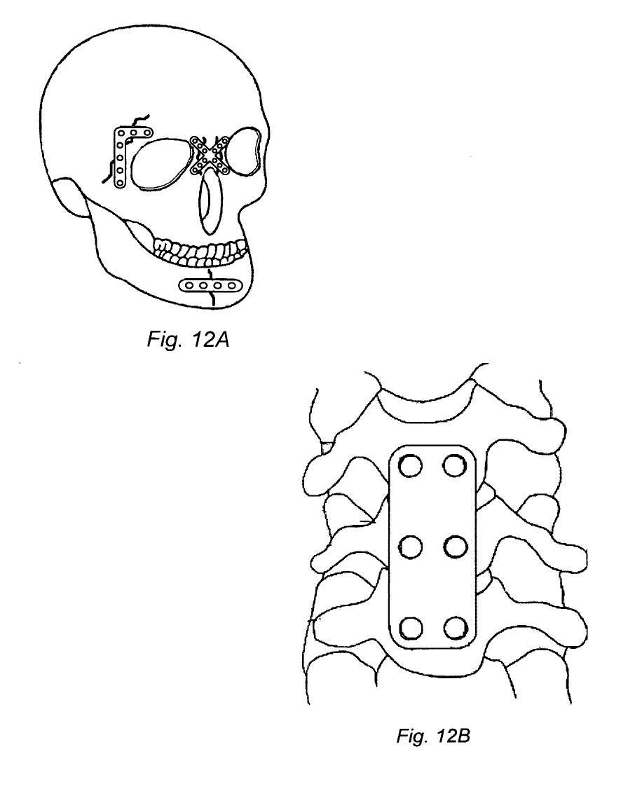

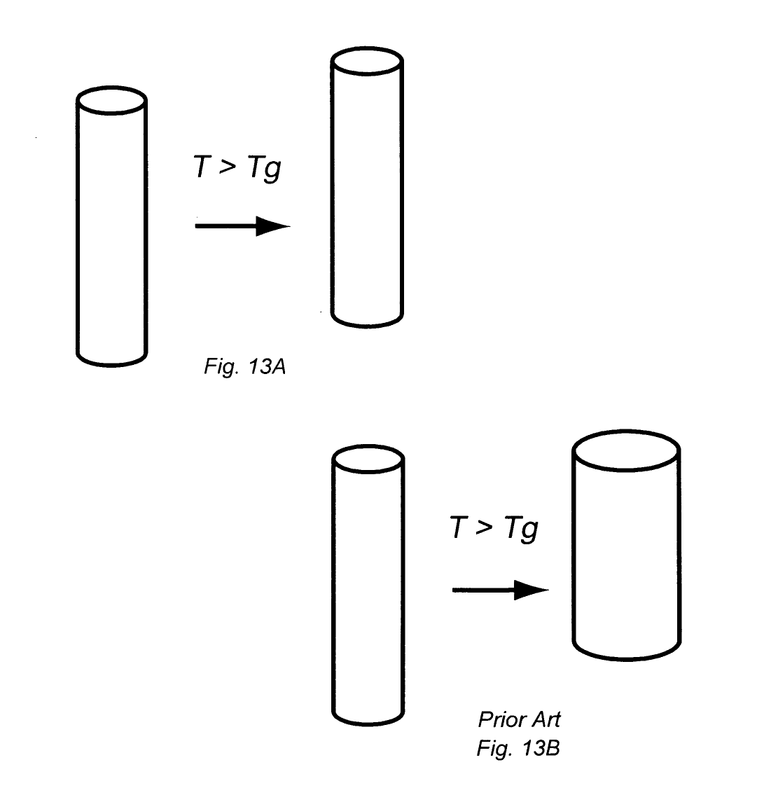

The finished device of this invention may be a craniomaxillofacial plate, such as those depicted in FIG. 12A, or an anterior cervical plate, such as that depicted in FIG. 12B. These cases illustrate the need for a high-strength device that is deformable to the complex geometry of the intended anatomical location. In order to achieve this deformation it is necessary to heat the device above its glass transition temperature. FIG. 13A illustrates that materials fabricated as described in the present invention are dimensionally stable above the glass transition temperature; that is, above Tg they maintain their size and shape without the need for externally applied constraints such as molds or clamps. This is significant because externally applied constraints may be cumbersome and unwieldy, and it is not clear that they would provide anything more than temporary maintenance of the size and shape. In contrasts, the prior art is depicted in FIG. 13B, as it illustrates that materials fabricated as described in the prior art are not dimensionally stable above the glass transition temperature, and thus lose their applicability for the intended use of the device.

### EXAMPLE 1

Rectangular slugs were fabricated by injection molding 85/15 poly(L-lactide-co-glycolide). The slugs were placed into a tooling arrangement heated to 96° C. and the polymer heated to a temperature above the Tg of the polymer but below the Tm of the polymer. A ram press was used to apply pressure to the slug and force the material into the die, the die having a smaller cross-section than the slug. A draw ratio of approximately 4:1 was used. The die geometry was such that it formed a straight plate approximately 50×7×2.5 mm (L×W×T). The slug and die were cooled to a temperature sufficient to allow removal of the pressed part. The parts were evaluated to determine their bending ability in hot water (65° C.). Previous trials with high-strength oriented polymer devices made by prior art continuous processes indicated that the material was highly unstable dimensionally when heated above its glass-transition to allow it to be bent. The high-strength, oriented plate of this example was found to be much easier to bend when heated and, surprisingly, it was found that the material inherently maintained its thickness and length.

### EXAMPLE 2

Rectangular slugs were fabricated by injection molding 85/15 poly(L-lactide-co-glycolide). The slugs were placed into a tooling arrangement heated to 110° C. and the polymer heated to a temperature above the Tg of the polymer but below the Tm of the polymer. A ram press was used to apply pressure to the slug and force the material into the die, the die having a smaller cross-section than the slug. A draw ratio of approximately 4:1 was used. The die geometry was such that it caused the material to curve at an angle creating a bent-axis L-plate. The slug and die were cooled to a temperature sufficient to allow removal of the pressed part. The parts were evaluated to determine their bending ability and L-plate geometry retention in hot water (65° C.). This temperature is well in excess of the polymer's Tg. The high-strength, oriented L-plate was found to be much easier to bend when heated and it was found that the material maintained its thickness and length, as well as its L-shape.

### EXAMPLE 3

Circular slugs were fabricated by extruding poly(L-lactide) rod and then cutting the rod to length using standard machining techniques. The slugs were placed into a tooling arrangement heated to 159° C. and the polymer heated to a temperature above the Tg of the polymer but below the Tm of the polymer. A ram press was used to apply pressure to the slug and force the material into the die, the die having a smaller cross-section than the slug. A draw ratio of approximately 4:1 was used. The die geometry was such that it formed a pin approximately 3.5×40 mm (Diameter x Length). The slug and die were cooled to a temperature sufficient to allow removal of the pressed part. The parts were evaluated to determine their dimensional and strength stability after heating in hot water (70 ° C.). This temperature is well in excess of the polymer's Tg. The parts were measured before and after immersion in hot water for 1 hour. An ANOVA analysis was performed and it was determined that there was no significant change in dimension due to hot water immersion (P(2-tail)>0.05). The samples were then shear tested and compared to other devices fabricated using the identical method. An ANOVA analysis was performed and it was determined that there was no significant change in shear strength due to hot water immersion (P(2-tail)>0.05).

### COMPARATIVE EXAMPLE 4

High-strength, oriented poly(L-lactide) pins were fabricated using a continuous drawing method such as that described in U.S. Pat. No. 6,719,935. A draw ratio of approximately 4:1 was used at a temperature of 182° C. The parts were evaluated to determine their dimensional and strength stability after heating in hot water (70° C.). This temperature is well in excess of the polymer's Tg. The parts were measured before and after immersion in hot water for 10 minutes. An ANOVA analysis was performed and it was determined that there was a significant change in dimension (increase of 7.85% in diameter and decrease of 11.58% in length) due to hot water immersion (P(2-tail)<0.05). It was also observed that the immersion in hot water resulted in a loss of straightness of the pin, as noticable warping or bending of the previously straight pins occurred when immersed in the heated water and heated above glass transition. The samples were then shear tested and compared to other devices fabricated using the identical method. An ANOVA analysis was performed and it was determined that there was a significant change (8.8% decrease) in shear strength due to hot water immersion (P(2-tail)>0.05). This comparative example indicates that the dimensional and strength stability of high-strength, oriented parts after exposure above their glass-transition is dependent on the manufacturing method.

The above described operational processes and practices may be performed to form an implantable device with zones of variable alignment of the polymer structure, zones of varying cross-section and preferably, final part geometry of the implantable device. Furthermore, the processes and practices described herein may be performed to form an implantable device with surprising dimensional stability, that provides for an oriented material that will retain its dimensions and degree of polymer orientation upon subsequent reheating to at least glass transition temperature.

Thus, since the invention disclosed herein may be embodied in other specific forms without departing from the spirit or general characteristics thereof, some of which forms have been indicated, the embodiments described herein are to be considered in all respects illustrative and not restrictive, by applying current or future knowledge. The scope of the invention is to be indicated by the appended claims, rather than by the foregoing description, and all changes which come within the meaning and range of equivalency of the claims are intended to be embraced therein.

What is claimed is:

1. A device suitable for implantation in a living being, said device comprising a hollow core tissue fixation device comprising an amorphous or at least partially crystalline polymer material that exhibits biaxial molecular orientation comprising at least a degree of molecular orientation arranged along an axis passing longitudinally throughout said polymer material, and also exhibiting at least a degree of molecular orientation arranged transverse to said axis, and wherein said device is made by the process of:
a. providing a polymer slug, die cavity tooling, and ram press, wherein said die cavity tooling defines a die shape having a plurality of zones of varying cross-section, and further wherein said die cavity tooling comprises a pin extending into a die cavity;
b. placing said polymer slug between said ram press and die cavity tooling;
c. heating at least said polymer slug to a temperature in a range between the glass transition temperature and the melting temperature;
d. after said heating, actuating said ram press in order to apply pressure upon said polymer slug, thereby deforming said polymer slug and forcing said polymer slug to conform to said die shape, wherein said deforming causes an alignment of said polymeric molecular structure along an axis, and further wherein said polymer slug deforms around said pin, thereby causing an alignment of said polymeric molecular structure around said pin and transverse to said axis; and
e. removing said device from said die cavity tooling.

2. The device made by the process of claim 1, the process further comprising the step of: machining said device to a finished product.

3. The device made by the process of claim 1, wherein said polymer slug comprises a resorbable polymer.

4. The device made by the process of claim 3, wherein said resorbable polymer is selected from the group consisting of PLA, PGA, PGA/PLLA, DLPLA, and combinations thereof.

5. The device made by the process of claim 1, wherein said polymer slug provided further comprises additive materials.

6. The device made by the process of claim 5, wherein said additive materials are selected from the group consisting of ceramics, fibrous materials, particulate materials, biologically active agents, plasticizers and combinations thereof.

7. The device made by the process of claim 1, wherein said die cavity tooling is temperature controlled.

8. The device made by the process of claim 1, wherein said barrel is temperature controlled.

9. The device made by the process of claim 1, wherein said ram press further comprises complex geometry.

10. The device made by the process of claim 1, wherein said die cavity tooling is not a single piece but rather comprises a plurality of pieces capable of fitting together.

11. The device made by the process of claim 1, wherein said polymer slug further comprises complex geometry.

12. The device made by the process of claim 1, wherein said die cavity tooling further comprises an ejection device.

13. The device made by the process of claim 12, wherein said ejection device comprises a pin.

14. The device made by the process of claim 12, wherein said ejection pin serves to form an end of said polymer slug.

15. The device made by the process of claim 1, arranged as a bone fixation device.

16. A device suitable for implantation in a living being, said device comprising a hollow core tissue fixation device, said device comprising an amorphous or at least partially crystalline polymer material that exhibits biaxial molecular orientation comprising at least a degree of molecular orientation arranged along an axis passing longitudinally throughout said device, and also exhibiting at least a degree of molecular orientation arranged transverse to said axis, said polymer material comprising at least first and second zones having cross section, wherein the polymer material in a first zone is more highly oriented than that polymer material in a second zone.

17. The device of claim 16, wherein said polymer material comprises a resorbable polymer.

18. The device of claim 17, wherein said resorbable polymer is selected from the group consisting of PLA, PGA, PGA/PLLA, DLPLA, and combinations thereof.

19. The device of claim 16 further comprising additive materials selected from the group consisting of ceramics, fibrous materials, particulate materials, biologically active agents, plasticizers and combinations thereof.
